(19) United States
(12) Patent Application Publication
(10) Pub. No.: US 2021/0363767 A1
(43) Pub. Date: Nov. 25, 2021

(54) Modular Protective Room and Pass-Through Hatch for a Protective Room

(71) Applicant: SEIWO Technik GmbH, Orebach (DE)

(72) Inventors: Jan Wabst, Chemnitz (DE); Mike Köhler, Mildenau (DE); Katrin Arnold, Burkhardtsdorf / OT Kemtau (DE)

(21) Appl. No.: 17/324,167

(22) Filed: May 19, 2021

(30) Foreign Application Priority Data
May 19, 2020 (DE) 102020113461.4
May 19, 2020 (DE) 202020102844.8

(51) Int. Cl. E04H 1/12; A47F 9/04; A61L 2/24; A61L 2/10; A61L 9/20; F24F 7/003; F24D 13/02

(52) U.S. Cl. CPC E04H 1/1277; A47F 9/04; A61L 2/24; E04B 1/24; A61L 9/205; F24F 7/003; F24D 13/024; A61L 2/10

(57) ABSTRACT

A modular shelter for one or more persons, comprising
at least one interior space which is closed off from at least one adjacent space by at least one circumferentially closed wall, a floor and a ceiling, wherein the circumferentially closed wall is penetrated by at least one closable access opening,
at least one presence sensor, which detects the presence and/or absence of a person in the interior space, and
at least one first irradiation device for irradiating the interior with UVC rays.

Furthermore, there is a kit for producing a modular shelter and a hatch for a shelter, comprising a partition wall and two opening flaps which are arranged on two different sides of the partition wall and which allow access to a sliding tray which is movable back and forth between the opening flaps.

### CROSS REFERENCE TO RELATED APPLICATIONS

This application claims the priority of DE 102020113461.4 filed on 2020 May 19, and DE 202020102844.8 filed on 2020 May 19; these applications are incorporated by reference herein in their entirety.

### BACKGROUND

The present invention relates to a modular shelter for one or more persons as well as a hatch for a shelter.

From appointments with notaries to the registration of a vehicle to the signing of a loan agreement—there are numerous appointments in public life that cannot be handled by video conferencing or a telephone call. Especially in view of possible pandemics, distance requirements and hygiene measures are becoming increasingly important. Offices, public administrations, service providers and many companies with frequent customer contact are thus faced with the challenge of protecting their employees and customers as best as possible against potential infection by viruses.

A promising starting point is the spatial isolation of persons who are unable to maintain a necessary minimum distance due to the situation.

Usually mobile room systems are pronounced as standard profile solutions and regular cubic shape. These are used indoors as meeting boxes or outdoors containers for meetings, stay or storage. The conventional room systems are not characterized by a controlled system of health protection systems in the form of light disinfection, air disinfection and antibacterial surfaces, as well as contactless control of the functions in the user area. As a rule, the conventional room systems have at most a heating or air conditioning unit, as well as a power supply for light and various consumers. Containment of a possible viral load thus appears difficult.

The publication DT 26 05 750 A1 describes a large container housing used and equipped as a mobile isolation station, comprising an isolation space separated from an entrance space. An antiviral and/or antibacterial treatment of the interior space is not provided, whereby microorganisms introduced by a user can multiply in an uncontrolled manner.

The publication DE 20 2005 008 334 U1 describes a cleanroom, in particular for a medical or biotechnological laboratory, which has an interior space and a supporting frame enclosing the interior space. Panels are pressed against support profiles of the supporting framework with the inclusion of sealing elements, resulting in a hermetic seal of the interior space. Here, too, antiviral and/or antibacterial treatment of the interior space is not provided.

The publication EP 1 588 720 A1 describes a mobile transport device designed for disinfecting, killing or passivating pathogens on organisms and/or objects, comprising a first section having at least one device for spraying a liquid containing an oxidative agent and a second section having at least one UV light source.

The publication EP 2 712 378 B1 describes a medical kiosk for remote consultation of a user located in an interior space of the medical kiosk, equipped with a video conferencing system and several medical compartments that can be locked remotely. Further, the medical kiosk has a cleaning or disinfection system, which may include a UV system. This has multiple UV lamps that irradiate the interior space in the absence of a user.

In the publication EP 3 087 227 B1, a building, in particular designed as a hospital, is disclosed, comprising a skeleton structure of several floors, wherein individual modules with one or more rooms are inserted into the skeleton structure. The hospital is supplied via a disinfecting airlock, for example an airlock comprising UV lamps. Disadvantageously, the known solutions are either not very flexible and in particular do not allow the use in connection with existing premises, or they do not provide sufficient disinfection of the interior space, or only insufficient protection against the spread of viruses and bacteria introduced by a user.

### SUMMARY

The invention is thus based on the object of depicting a flexible, modular, sustainably recyclable room system that protects the health of users and operators in the best possible way.

This object is solved on the one hand by a modular shelter for one or more persons, comprising
at least one interior space which is closed off from at least one adjacent space by at least one circumferentially closed wall, a floor and a ceiling, wherein the circumferentially closed wall is penetrated by at least one closable access opening,
at least one presence sensor, which detects the presence and/or absence of a person in the interior space, and
at least one first irradiation device for irradiating the interior with UVC rays.

### DETAILED DESCRIPTION

The interior space is accessible from at least one side and forms a space closed on all sides in the exterior and a space closed on at least five sides in the interior space.

Advantageously, the modular shelter according to the invention is designed in a regular cubic shape. Alternatively, the modular shelter according to the invention can also represent curved or organic spatial shapes.

Preferably, the modular shelter according to the invention has at least one air purification system for filtering the room air located in the at least one interior space.

For this purpose, the room air is advantageously drawn in by an element for drawing in the exhaust air, then filtered and/or irradiated and/or purified in some other way, and finally released back into the interior via an element for discharging the purified air.

Alternatively, it is possible to discharge the extracted exhaust air either in purified or in unpurified form into the adjacent outdoor space and to draw in fresh air from the outdoor space, which can then optionally be filtered and/or irradiated and/or otherwise purified and finally discharged back into the indoor space via an element for discharging the purified air.

Advantageously, the presence sensor is a motion detector, an optical sensor, a heat sensor, a camera, or a capacitive sensor, or at least two of the aforementioned sensors are combined.

The presence sensor can be placed directly at the access opening. Inevitably, however, the presence sensor is arranged so that it is able to detect whether one or more persons are present in the interior space of the modular shelter.

The modular shelter according to the invention further comprises a control unit which controls the existing irradiation devices and/or the access opening and/or the air purification system and further electronic components and which detects signals from the presence sensor and from possible further sensors, for example a temperature sensor. The control unit is preferably an intelligent control unit, wherein predetermined routines can also be considered. A heating system, for example, can also be controlled by the control unit.

In particular, when the presence of at least one person in the interior space is detected by the presence sensor, the at least one first irradiation device irradiates the interior space with UVC radiation having a wavelength of 100 nm to 230 nm, preferably 222 nm.

Irradiation with such a wavelength can advantageously be used as a permanent background irradiation, since it already has an antiviral and antibacterial effect, but at the same time does not have a harmful effect on persons present in the indoor space.

Shaded areas in the interior, for example in the region of cabinets, tables and the like, are preferably lined with additional UVC lamps to ensure optimum irradiation and prevent the accumulation of pathogens/viruses and/or bacteria.

At the same time, when one or more persons enter the interior space, interior lighting with light in the visible wavelength range can be switched on automatically. This light can be emitted, for example, by area lights, recessed ceiling spots and/or surface-mounted lighting units.

Preferably, the modular shelter according to the invention comprises at least a second irradiation device which irradiates the interior with UVC radiation having a wavelength above 222 nm in case of an absence of persons in the interior detected by the presence sensor.

Preferably, the second irradiation device irradiates the interior space with light having a wavelength of 250 nm to 290 nm.

Preferably, the modular shelter according to the invention has a timer which switches off the at least one second irradiation device after a predetermined time interval, preferably after one to four minutes, particularly preferably after 2 minutes.

The timer is advantageously coupled to the control device.

In particular, the modular shelter according to the invention has an access interlock system that blocks the access opening for the duration of the irradiation by the at least one second irradiation device.

This ensures that no person comes into contact with the potentially harmful UVC radiation in the higher wavelength range.

The irradiation time by the second irradiation device can be indicated by a signal lamp on the outside of the modular shelter according to the invention.

Advantageously, a motion-controlled and/or signal-controlled automatic door and/or a door with mechanical locking is arranged in the at least one lockable access opening.

Access is preferably contactless, for example by voice input, radio transmitter, RFID transmitter or other contactless opening or closing.

Access is also preferably designed to be barrier-free.

The access opening can alternatively be equipped with a standard manually operated door.

A further disinfection unit, for example for dispensing a hand disinfectant, can also be arranged in the area of the access opening.

Preferably, surfaces located in the interior space are formed of an antibacterial and/or antivirulence material and/or are coated with an antibacterial and/or antivirulence material.

This applies in particular to surfaces within reach of the user, such as integrated tables or panels.

In particular, an antibacterial and/or antivirulence material is selected from stainless steel, aluminum brass, plastic, ionized plastic, composite materials consisting of plastic and mineral components, wood-based material with a coating of antibacterial and/or antivirulence material, or any combination of the foregoing.

Preferably, the air purification system has a third air irradiation device, wherein the room air is sucked in by the air purification system, the aspirated room air is irradiated by the air irradiation device with UVC radiation having a wavelength of from 230 nm to 280 nm, preferably from 254 nm to 265 nm, and the irradiated room air is discharged back into the interior space by the air purification system.

Alternatively or additionally, other filter methods are also conceivable for filtering the aspirated exhaust air.

Alternatively, the air purification system can draw in outside air, irradiate it and release it into the interior space.

In a preferred embodiment, the modular shelter according to the invention has at least one interior wall for separating a first interior space from a second interior space or for separating the interior space from an existing building.

The modular shelter according to the invention can also be attached to an exterior or interior wall of an existing building, for example.

Advantageously, a first person can stay in the first interior space and a second person in the second interior space, which can thus communicate with each other in a protected environment. For this purpose, at least a partial area of the inner wall is of transparent and/or translucent design.

The modular shelter may be equipped with an intercom and/or with an audio system so that communication between the two communicators is ensured.

Advantageously, the modular shelter according to the invention has at least one electromechanical and/or mechanical hatch.

The hatch advantageously has a partition wall and two opening flaps, which are arranged on two different sides of the partition wall and which allow access to a sliding tray that can be moved back and forth between the opening flaps. The partition wall is in particular of transparent and/or translucent design.

Preferably, at least one surface irradiation device and/or at least one tray irradiation device for irradiating the interior space of the sliding tray with UVC rays is/are arranged in an interior space of the sliding tray.

Advantageously, the sliding tray can be moved electromechanically and/or mechanically. For this purpose, an actuating lever and/or switch can be arranged on one or both sides of the hatch.

Preferably, a bottom surface of the sliding tray is formed of UV-transmissive glass or UV-transmissive plastic.

An object or document placed in the sliding tray is irradiated with UVC rays after the opening flaps are closed and thus also cleaned of microorganisms.

Preferably, the at least one surface irradiation device and/or the at least one tray irradiation device irradiate the interior space of the sliding tray with UVC radiation having a wavelength above 222 nm when the opening flaps are closed.

The control of the sliding tray including its irradiation devices may be coupled to the control unit of the modular shelter according to the invention.

The hatch can be arranged in the wall of the modular shelter or in an inner wall of the modular shelter. The use of several hatches in a modular shelter is also conceivable.

The hatch can also be equipped without a disinfection system or with an alternative disinfection system.

Preferably, the modular shelter according to the invention comprises a temperature sensor for detecting a body temperature of at least one person located in the modular shelter.

In the event of an increased body temperature, which could indicate a risk of infection, a visual and/or audible warning signal can be emitted. Guide values for a “regular” body temperature can be programmed in the control unit or be pre-programmed.

Further preferably, the modular shelter according to the invention has a screen and/or a video conferencing system and/or a camera and/or a scanning unit and/or a printer and/or an intercom system and/or an audio system and/or one or more interfaces for a projector, a laptop, a scanner and/or a printer.

Advantageously, the modular shelter according to the invention has a heating system, in particular a raised floor with integrated infrared heating.

A modular shelter equipped with a raised floor is particularly suitable for outdoor use. The room system for outdoor use preferably has a rain-, water-, storm-, snow- and/or sun-proofed thermally insulated ceiling and can also be designed to be vandalism-proof.

In particular, a plurality of support profiles aligned perpendicularly to the frame profile are arranged or can be fitted on a circumferential frame profile, wherein at least two suspension profiles are fitted or can be fitted on one support profile in each case, wherein at least one filling element is fitted or can be fitted on each suspension profile.

The system for the interior space is preferably single-walled, but can also be double-walled. It preferably has an interior ceiling with the possibility of integrating lighting units (e.g. surface lights, recessed ceiling spots, surface-mounted lighting units and the UVC disinfection light or monitors or running lights, projectors).

Preferably, a suspension profile is hooked in or can be hooked in by means of at least one recess in a shaped element which is arranged or can be attached in a support profile facing radially outwards with respect to the support profile.

Alternatively or additionally, a connection can be made by means of screws, rivets, welding or the like.

Hooking in simplifies assembly and disassembly and allows for quick, situational modification of the modular shelter.

Preferably, the at least one filling element is selected from a material selected from metal, wood-based materials, plastics, composite elements, mineral materials, acrylic mineral materials, transparent or translucent materials, integrated screens, and/or front-projecting monitors.

In particular, a transparent or translucent material is glass, switchable glass, and/or plastic.

Advantageously, the glass is safety glass and the glass consists of a single-layer safety glass pane or a multilayer safety glass laminated pane.

In addition to the one or more irradiation unit(s) for irradiating with UVC light, further irradiation units for illuminating the interior with light in the visible wavelength range can be provided.

A signal light may further be arranged on the exterior wall near the access opening to indicate whether one or more persons are present in the interior space.

The object is further solved by a kit for the production of a modular shelter according to the invention, comprising at least one frame profile or at least two frame profile elements connectable to a frame profile, at least three support profiles, at least six suspension profiles, at least three filling elements and at least one ceiling element.

Preferably, the kit according to the invention for producing a modular shelter according to the invention comprises at least one presence sensor which detects the presence and/or absence of a person in an interior space of the modular shelter, and at least one first irradiation device for irradiating the interior space with UVC rays.

In particular, the kit according to the invention for producing a modular shelter according to the invention further comprises an air purification system for filtering the room air located in the at least one interior space.

The air purification system preferably includes at least one element for aspirating the room air located in the interior space and one element for discharging fresh air into the interior space. Furthermore, elements for irradiating the aspirated room air with UVC rays and/or filter elements are preferably included.

All components described above in the context of the description of the modular shelter according to the invention can also be included in the kit for manufacturing a modular shelter according to the invention.

The object is further solved by a kit for converting existing rooms into a modular shelter according to the invention, wherein the kit comprises at least one UVC lamp and at least one presence sensor.

Preferably, the kit according to the invention further comprises a control unit for converting existing rooms into a modular shelter according to the invention.

The control unit is advantageously designed as an intelligent control system.

All components described above in the context of the description of the modular shelter according to the invention can also be included in the kit according to the invention for converting existing rooms into a modular shelter according to the invention.

The object is further solved by a hatch for a shelter comprising a partition wall and two opening flaps which are arranged on two different sides of the partition wall and which allow access to a sliding tray movable to and fro between the opening flaps, wherein according to the invention at least one surface irradiation device and/or at least one tray irradiation device for irradiating the interior of the sliding tray with UVC rays is/are arranged in an interior space of the sliding tray.

In particular, the partition wall is of transparent and/or translucent design.

Advantageously, the sliding tray can be moved electromechanically and/or mechanically.

For this purpose, an operating lever and/or switch can be arranged on one or both sides of the hatch.

Preferably, a bottom surface of the sliding tray is formed of UV-transmissive glass or UV-transmissive plastic.

Preferably, the at least one surface irradiation device and/or the at least one tray irradiation device irradiate the interior of the sliding tray with UVC radiation having a wavelength above 222 nm when the opening flaps are closed.

The modular shelter according to the invention, as well as the kits and the hatch according to the invention, advantageously ensure communication between people in public areas that is virtually free of bacteria and viruses.

Due to the use of UVC light, viruses are completely destroyed when used for a correspondingly long time. With UVC light at a lower dose, the virus load is kept so low that there is no longer any danger to health. Added to this is the use of antibacterial surfaces on the work areas and in areas with physical contact (table and seat surfaces, handles). These surfaces minimize the growth of microorganisms. The use of disinfectants that attack surfaces or endanger health in higher concentrations can be dispensed with. By using the modular shelter, the kits or the hatch, it is possible to dispense with wearing mouth and nose protection in the rooms, which in turn has a positive effect on the discussion situation, the work results and the psyche of all participants.

Advantageously, the solutions according to the invention are designed for non-contact control or use.

Use of the proposed solutions is conceivable, for example, in retirement homes, public authorities, law firms and other offices with frequent customer contact, in meeting situations or at trade fairs. The systems can also be used in the entrance area of major events such as concerts, trade fairs, soccer matches or supervisory board meetings.

### BRIEF DESCRIPTION OF THE DRAWINGS

The invention is explained in more detail below with reference to exemplary embodiments and associated figures, without being limited thereto, wherein:

FIG. 1 shows a schematic representation of an interior space of a modular shelter according to the invention;

FIG. 2 shows a floor plan of an embodiment of a modular shelter according to the invention with an interior space;

FIG. 3 shows a floor plan of an embodiment of a modular shelter according to the invention with two interior spaces;

FIG. 4 shows a floor plan of a further embodiment of a modular shelter according to the invention;

FIG. 5 shows a floor plan of a further embodiment of a modular shelter with video conferencing system according to the invention;

FIG. 6 shows a floor plan of a further embodiment of a modular shelter according to the invention with multiple access points;

FIG. 7 shows a schematic view of a hatch of a modular shelter according to the invention;

FIG. 8 shows a longitudinal section through a hatch of a modular shelter according to the invention;

FIG. 9 shows an exploded view of the connection area between the support profile and frame profile of a modular shelter according to the invention;

FIG. 10 shows a cross-section through a wall area of a modular shelter according to the invention;

FIG. 11 shows a schematic view of the connection area between the support profile and the suspension profile of a modular shelter according to the invention;

FIG. 12 shows a sectional view of a wall area of a modular shelter according to the invention intended for indoor use;

FIG. 13 shows a sectional view of a wall area of a modular shelter according to the invention intended for outdoor use with a visible support profile; and

FIG. 14 shows a sectional view of a wall area of a further modular shelter according to the invention with invisible support profile intended for outdoor use.

### DETAILED DESCRIPTION OF THE PREFERRED EMBODIMENTS

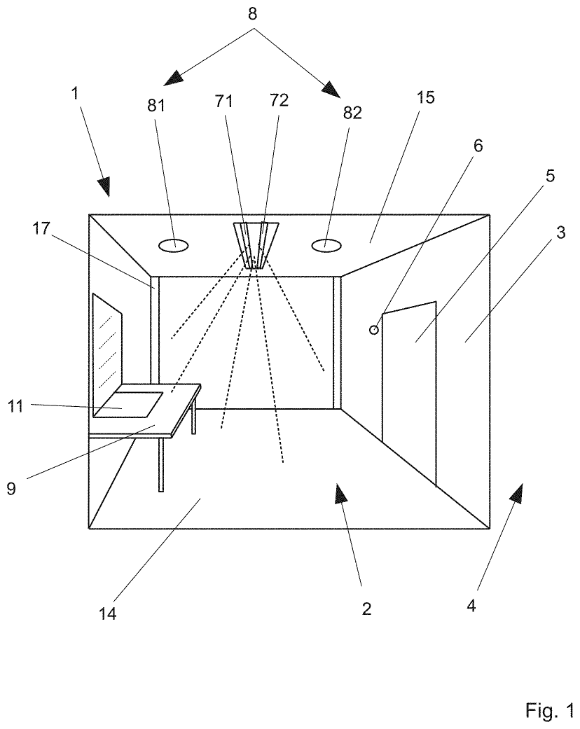

FIG. 1 shows a schematic representation of an interior space 2 of a modular shelter 1 according to the invention. The interior space 2 is surrounded by a circumferentially closed wall 3, which is penetrated only by an access opening 5 and separates the interior space 2 from an adjacent space 4. The modular shelter 1 further has a floor 14 and a ceiling 15, wherein the ceiling 15 comprises a plurality of inlets for irradiation devices 71, 72 and an air purification system 8.

In the embodiment shown, a first irradiation device 71 is designed as a UV LED lamp and emits UVC radiation with a wavelength of approximately 222 nm into the interior space 2 when at least one person is present in the interior space 2. The presence of a person is thereby detected by means of a presence sensor 6, which may be a motion detector, for example. In the present exemplary embodiment, the presence sensor 6 is arranged in the wall 3; however, it can also be located in the ceiling 15 or at another position from which it can detect the interior space 2.

In the embodiment shown, a second irradiation device 72 is also designed as a UV LED lamp and emits UVC radiation with a wavelength of 250 nm to 290 nm into the interior space 2 when a person is absent from the interior space 2. The absence of a person is also detected by the presence sensor 6. After the person leaves the room, the room is electronically blocked for at least 2 minutes and the UVC light (with a frequency between 250 and 290 nanometers) is switched on and disinfects the room and the surfaces from bacteria and viruses.

The power consumption of the second irradiation device 72 is between 10 and 25 W and is possible for rooms up to 15 sqm. Larger rooms require supplementary UVC light sources. One light source can be used per 15 sqm interior space 2 as a fluorescent tube or a full or partial LED UVC light. Additional furniture or fixtures are illuminated with additional UVC light sources to compensate for any shadowing. Any cubature of the room as well as any interior fixtures will be taken into account.

A preferably automatic door is provided in the access opening 5, which can be opened via a further motion detector (not shown). Preferably, the shelter has an access locking system, also not shown, which locks the door when the interior space 2 is irradiated by the second irradiation device 72, so that unintentional entry by a user is prevented. The duration of the irradiation by the second irradiation device 72 as well as the access locking can be controlled via a timer which is also not shown.

Optionally, further lighting devices not shown may be provided in the ceiling 15 for irradiating the interior space 2 with visible light.

In the embodiment shown, the air purification system 8 has an element for aspirating the exhaust air 81 and an element for discharging the supply air 82. The aspirated exhaust air is irradiated with UVC radiation with a wavelength of 254 nm to 265 nm by means of a third irradiation device (not shown). Alternatively or additionally, other filtering methods for filtering the intake exhaust air are also conceivable. In a preferred embodiment, the power consumption of the third irradiation device is 20-135 W; 55-350 m³/h. The devices can be adapted to the room size.

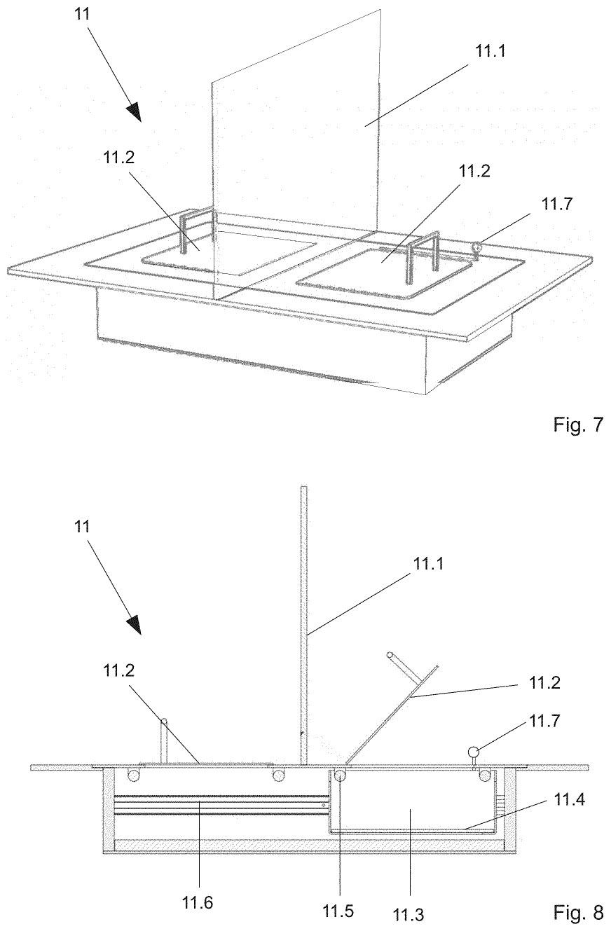

In the interior space 2, a table top is also present in the illustrated exemplary embodiment, the surface 9 of which is coated with an antivirulence material. Other surfaces not shown, such as chairs and/or technical equipment, may also be present and are preferably also coated with an antibacterial and/or antivirulence material or made of such a material. Integrated into the table top is a hatch 11, the precise operation of which is explained in FIGS. 7 and 8.

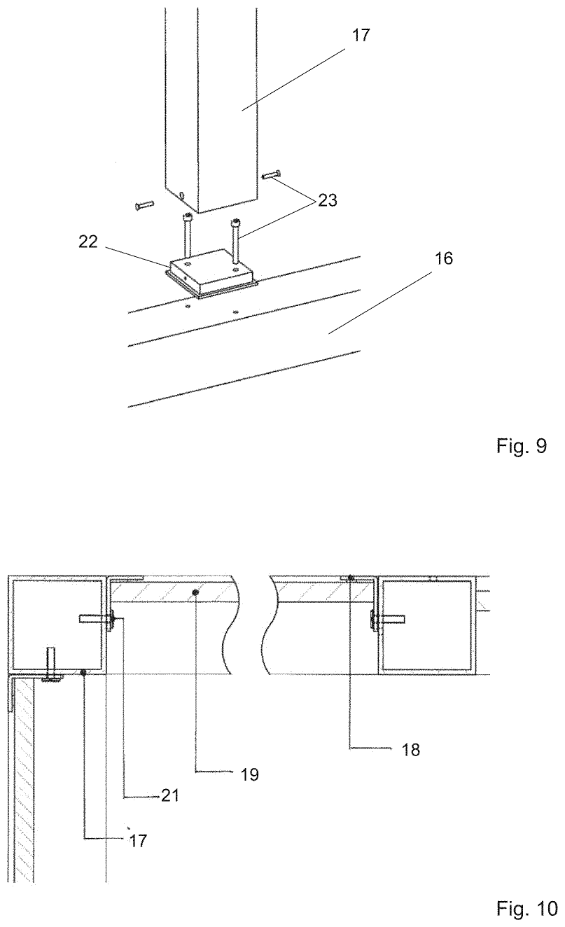

Also visible are support profiles 17, whose interaction with other elements is explained in FIGS. 9 to 14.

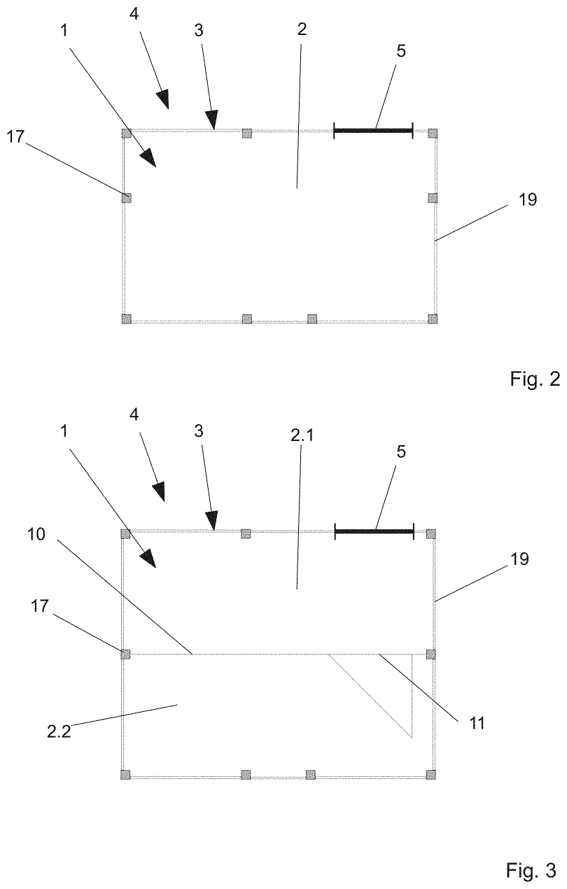

FIGS. 2 to 6 show floor plans of various embodiments of the modular shelter 1 according to the invention. The circumferentially closed wall 3 is always formed by support profiles 17, filling elements 19 and suspension profiles (not shown). The support profiles 17 are arranged in a fixed grid on a frame profile. In all embodiments of FIGS. 2 to 6, the wall 3 is penetrated by at least one access opening 5 in which a lockable, automatically controlled door is preferably arranged.

A one-room system as a digital media room is used for handling official procedures with a direct communication system or a communication system guided by software algorithms through an audio-visual interface (e.g. screen with camera and audio system, projector with camera or motion sensors and audio output/audio input); in addition, the single room can be supplemented with further technology for printing, copying, document exchange (e.g. hatches or mailboxes), audio input, gesture control. The room is closed on at least five sides with at least one access inside buildings, and outside buildings as an outdoor solitary variant at least six sides closed with at least one access. Basically, the single room is provided with at least one passive disinfection unit by means of UVC light, double-stage UVC light or/and air disinfection. Furthermore, the one-room system can be equipped with a mechanical or electronic, controllable or non-controllable locking system. An electronic access control system enables scheduling and occupancy planning. Access is via a security door using a latch control with antibacterial latch (preferably stainless steel) or via an automatic door using electronic access, by code entry or contactless. The room system can be transported individually in components or pre-assembled as a whole. The dimensions may vary, and the room may be a cuboid, cube, oval, round or otherwise shaped.

In the embodiment shown in FIG. 3, an interior wall 10 separates a first interior space 2.1 from a second interior space 2.2. A hatch 11 is arranged between the first interior space 2.1 and the second interior space 2.2. A visitor can enter the first interior space 2.1 through the access opening 5 and preferably communicate with a person located in the second interior space 2.2, for example an employee or a consultant, through a security glass pane and intercom. Documents or smaller items can be exchanged through the hatch 11. Access to the second interior space 2.2 can be gained via the wall 3 or the interior wall 10.

In principle, antibacterial surfaces or normal surface coatings are also used in the interior of the two-room or double-room system. The room is closed on all sides and has a physical room separation in the middle or offset. The room separation may be partially or fully glazed or closed with other materials. The double-room system is equipped with an intercom system or a similarly functioning audio system and is used for communication without establishing physical contact. In addition, the system is equipped with one or more disinfection systems (such as UVC, double-level or multi-level UVC light 222 nm and/or 250 to 280 nm UVC light, air disinfection) on both sides. Thus, the room offers a minimum risk of infection. Each room is accessed separately via a door, which can also preferably be operated as an electronically controlled automatic door. One room serves as a customer room and the opposite one as an administration room. The administrator can control all functions in the customer room manually. Both rooms can be equipped with office communication systems, such as printer, copier, scanner. In addition, a hatch can be installed between the rooms for the exchange of documents and/or goods with and without a disinfection unit (e.g. by UVC light).

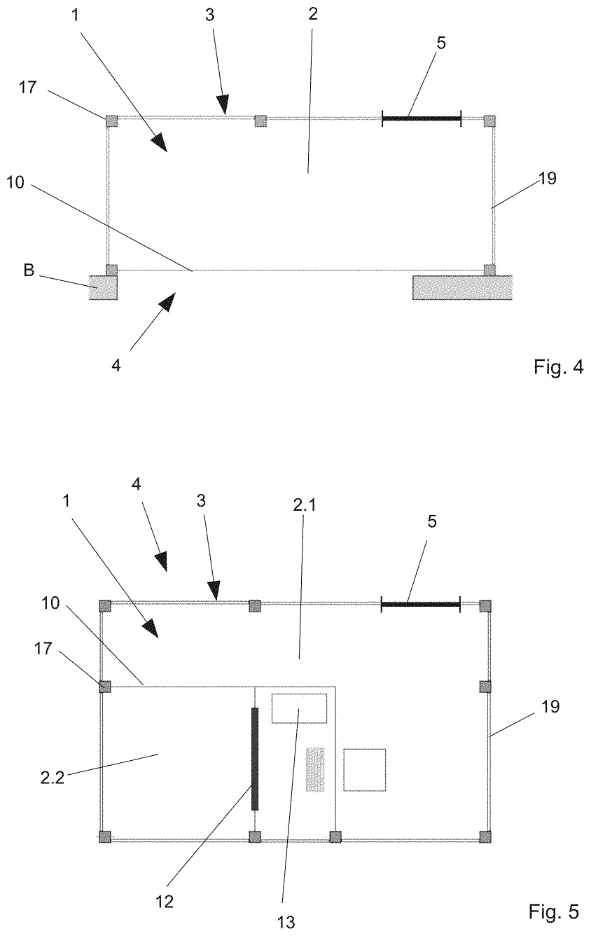

FIG. 4 shows an embodiment of a modular shelter 1, which is arranged on a wall of an existing building B. This can be an exterior wall or an interior wall of an existing building B. Separation of the interior space 2 from an exterior space 4 is achieved here, for example, by attaching the shelter 1 to an exterior facade of a building B, with a secure seal to the building B being provided, for example, by appropriate sealing materials such as silicone, expanding tape and the like.

A person located in the exterior space 4 can thereby communicate with a person located in the interior space 2 also through a safety glass pane and intercom. A hatch (not shown) can also be present.

The embodiment shown in FIG. 5 also shows a modular shelter in which a first interior space 2.1 is separated from a second interior space 2.2 by an interior wall 10. The second interior space 2.2 can be used, for example, as a technical space. The first interior space 2.1 contains, among other things, a video conferencing system 12 with a screen and camera, as well as other equipment, such as printers 13, scanners and/or input devices. The dimensions of such a modular shelter can be given as 4 m in length, 3 m in width and 2.5 m in height as an example.

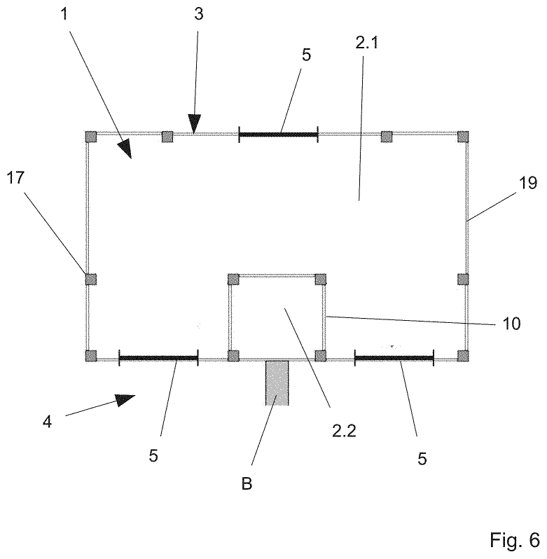

FIG. 6 shows an embodiment which, for example, is arranged in the interior space of a building or of a room of a building in the case of a limited exterior construction space. Here, too, a first interior space 2.1 is separated from a second interior space 2.2 by an interior wall 10, which can be used as a technical space. The modular shelter 1 has three access openings 5, one of which could be used, for example, as an entrance and two as an exit, or two as an entrance and one as an exit, in order to guide a controlled flow of visitors into or out of an existing building B. There could also be hatches, for example to outside ticket booths.

FIG. 7 shows a schematic view of a hatch 11 of a modular shelter according to the invention. The hatch 11 can be arranged between a first interior space and a second interior space or between an interior space and an exterior space of the modular shelter. It has a separating pane 11.1 through which two persons using the hatch 11 can see each other. Advantageously, an intercom system not shown is also provided. On each side of the separating pane 11.1 there is an opening flap 11.2 which allows access to a sliding tray located underneath. An actuating lever 11.7 is arranged on one side of the separating pane 11.1, by means of which the sliding tray can be moved to the opposite side and back. A second actuating lever may additionally be provided on the opposite side. All surfaces of the hatch are coated with an antibacterial and/or antivirulence material or are formed from such a material.

The longitudinal section through a hatch 11 of a modular shelter according to the invention shown in FIG. 8 illustrates how the hatch 11 functions. After opening an opening flap 11.2, a document or smaller object can be inserted into the sliding tray 11.3. The opening tray is then closed and a surface irradiation device 11.4 irradiates the object/document located in the tray from the underside with UVC rays having a wavelength of 254 nm, while a plurality of tray irradiation devices 11.5 irradiate the object/document located in the tray from above with UVC rays. The bottom of the sliding tray 11.3 is advantageously formed of UV-transmissive material, preferably UV-transmissive glass. The user can push the sliding tray 11.3 to the opposite side of the separating pane 11.1 by means of the actuating lever 11.7, wherein the sliding tray 11.3 is guided along guide rails 11.6. A second user can now open the opening flap 11.2 on his side of the separating pane 11.1 and remove the disinfected items/documents.

The compartment size of the sliding tray 11.3 can be variable. As an alternative to the mechanical movement by an actuating lever 11.7 shown here, the sliding tray 11.3 can be moved electrically.

The surface irradiation device 11.4 and the tray irradiation devices 11.5 are designed in such a way that irradiation of the interior of the sliding tray 11.3 takes place only when the opening flaps 11.2 are closed.

FIG. 9 shows an exploded view of the connection area between the support profile 17 and the frame profile 16 of a modular shelter according to the invention. An adapter element 22 is attached to the frame profile 16 by means of screws 23. A support profile 17 is placed on the adapter element 22 and also fastened by means of screws 23. The media (network and power cables) are pre-installed in the profile or outside in protected channels and ready to plug in (with intermediate coupling).

FIG. 10 shows a cross-section through a wall area of a modular shelter according to the invention. A suspension profile 18 is attached to a support profile 17 by means of a shaped element 21. In this context, a plurality of suspension profiles 18 can be arranged on a support profile 17. Each suspension profile 18 is connected to a filling material 19, wherein the connection between suspension profile 18 and filling material 19 can be made by gluing, welding, riveting or similar fastening methods, depending on the material.

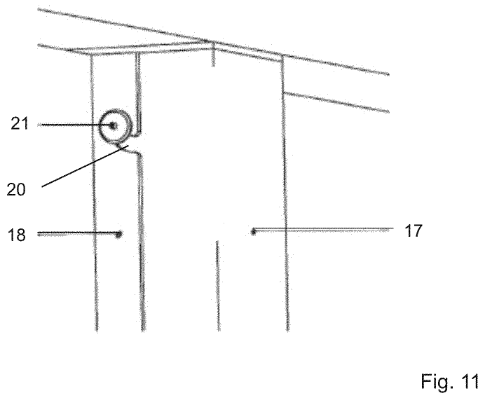

FIG. 11 shows a schematic view of the connection area between the support profile 17 and the suspension profile 18 of a modular shelter according to the invention. A shaped element 21, for example a screw, is arranged in the support profile 17 and faces radially outwards with respect to the support profile 17. However, the shaped element 21 may also be a pin. The associated suspension profile 18 has a corresponding tongue-shaped recess 20, via which the suspension profile can be hooked onto the shaped element 21. The connection is stiffened by tightening the screw (shaped element 21). Advantageously, the construction can be easily assembled and disassembled.

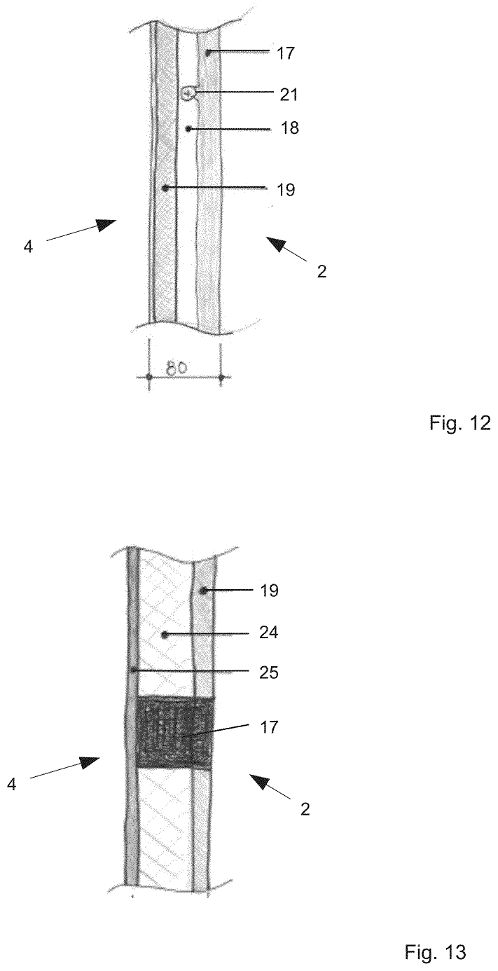

FIG. 12 shows a sectional view of a wall area of a modular shelter provided for indoor use according to the invention. The wall area delimits an outer area 4 from the inner area 2 of the modular shelter. The support profile 17 is an 80 cm×80 cm aluminum profile. A suspension profile 18 is suspended via a shaped element 21, to which a filling material 19 is attached.

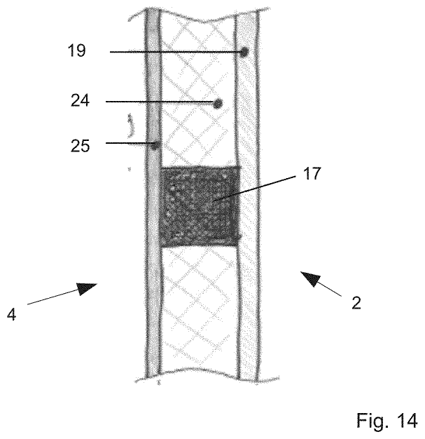

FIG. 13 shows a sectional view of a wall area of a modular shelter according to the invention intended for outdoor use with a visible support profile 17, FIG. 14 shows a sectional view of a wall area of another modular shelter according to the invention intended for outdoor use with an invisible support profile 17.

In the embodiment shown in FIG. 13, the filling material 19 is attached to the support profile 17 (via a suspension profile not shown) in such a way that the support profile is visible from the interior space 2. An outer cladding 25 is attached to the outer area 4. A thermal insulation 24 is arranged between the outer cladding 25 and the filling material 19.

In the embodiment shown in FIG. 14, the filling material 19 is attached to the support profile 17 (via a suspension profile not shown) in such a way that the support profile is not visible from the interior space 2. An outer cladding 25 is attached to the outer area 4. A thermal insulation 24 is arranged between the outer cladding 25 and the filling material 19.

In both variants, the support profile 17 is made of wood with dimensions of 80 cm×100 cm.

### LIST OF REFERENCE NUMERALS

1 Modular shelter
2 Interior space
2.1 First interior space
2.2 Second interior space
3 Wall
4 Adjacent space
5 Access opening
6 Presence sensor
71 First irradiation device
72 Second irradiation device
8 Air purification system
81 Exhaust air
82 Supply air
9 Surface
10 Interior wall
11 Hatch
11.1 Separating pane
11.2 Opening flap
11.3 Sliding tray
11.4 Surface irradiation device
11.5 Tray irradiation device
11.6 Guide rail
11.7 Actuating lever
12 Video conferencing system
13 Printer
14 Floor
15 Ceiling
16 Frame profile
17 Support profile
18 Suspension profile
19 Filling element
20 Recess
21 Shaped element
22 Adapter element
23 Screws
24 Thermal insulation
25 Exterior cladding
B Existing building

1. A modular shelter (1) for one or more persons, comprising
at least one interior space (2), which is closed off from at least one adjacent space (4) by at least one circumferentially closed wall (3), a floor (14) and a ceiling (15), wherein the circumferentially closed wall (3) is penetrated by at least one closable access opening (5),
at least one presence sensor (6), which detects the presence and/or absence of a person in the interior space (2), and
at least one first irradiation device (71) for irradiating the interior space (2) with UVC radiation, wherein the at least one first irradiation device (71) irradiates the interior space with UVC radiation having a wavelength of 100 nm to 230 nm when the presence of at least one person in the interior space (2) is detected by the presence sensor (6),
at least one second irradiation device (72) which, in the event of an absence of persons in the interior space (2) detected by the presence sensor, irradiates the interior space (2) with UVC radiation having a wavelength above 222 nm, wherein during the duration of the irradiation by the at least one second irradiation device (72) an access locking system blocks the access opening (5),
at least one air purification system (8) for filtering the room air located in the at least one interior space (2), wherein the air purification system (8) comprises a third air irradiation device and wherein the room air is aspirated by the air purification system (8), the aspirated room air is irradiated by the air irradiation device with UVC radiation having a wavelength of 230 nm to 280 nm, and the irradiated room air is discharged again into the interior space by the air purification system (8),
surfaces (9) located in the interior space, which are formed from an antibacterial and/or antivirulence material and/or are coated with an antibacterial and/or antivirulence material.

2. The modular shelter (1) according to claim 1, characterized in that the presence sensor (6) is a motion sensor, an optical sensor, a thermal sensor, a camera or a capacitive sensor, or in that at least two of the aforementioned sensors are combined.

3. The modular shelter (1) according to claim 1, characterized in that the at least one first irradiation device (71) irradiates the interior space with UVC radiation having a wavelength of 222 nm upon a presence of at least one person detected by the presence sensor (6).

4. The modular shelter (1) according to claim 1, comprising a timer which switches off the at least one second irradiation device (72) after a predetermined time interval, preferably after one to four minutes, particularly preferably after 2 minutes.

5. The modular shelter (1) according to claim 1, characterized in that a motion-controlled and/or signal-controlled automatic door and/or a door with mechanical locking is arranged in the at least one lockable access opening (5).

6. The modular shelter (1) according to claim 5, characterized in that an antibacterial and/or antivirulence material is selected from stainless steel, aluminum brass, plastic, ionized plastic, composite materials consisting of plastic and mineral components, wood-based material having a coating of antibacterial and/or antivirulence material, or any combination of the aforementioned materials.

7. The modular shelter (1) according to claim 1, characterized in that the air purification system (8) irradiates the aspirated room air by the air irradiation device with UVC radiation having a wavelength of 254 nm to 265 nm

8. The modular shelter (1) according to claim 1, comprising at least one inner wall (10) for separating a first inner space (2.1) from a second inner space (2.2) or for separating the inner space (2) from an existing building (B).

9. The modular shelter (1) according to claim 1, comprising at least one electromechanical and/or mechanical hatch (11).

10. The modular shelter (1) according to claim 1, comprising a temperature sensor for detecting a body temperature of at least one person located in the modular shelter.

11. The modular shelter (1) according to claim 1, further comprising a screen and/or a video conferencing system (12) and/or a camera and/or a scanning unit and/or a printer (13) and/or an intercom system and/or an audio system and/or one or more interfaces for a projector, a laptop, a scanner and/or a printer.

12. The modular shelter (1) according to claim 1, comprising a heating system, in particular a raised floor with integrated infrared heating.

13. The modular shelter (1) according to claim 1, characterized in that a plurality of support profiles (17) aligned perpendicularly to the frame profile are arranged on a circumferential frame profile (16), and in that at least two suspension profiles (18) are attached to a respective support profile (17), wherein at least one filling element (19) is on each suspension profile (18).

14. The modular shelter (1) according to claim 13, characterized in that a suspension profile (18) is suspended by means of at least one recess (20) in a shaped element (21) arranged in a support profile (17) facing radially outwards with respect to the support profile (17).

15. The modular shelter (1) according to claim 13, characterized in that the at least one filling element (19) is selected from a material selected from metal, wood materials, plastics, composite elements, mineral materials, acrylic mineral materials, transparent or translucent materials, integrated screens and/or front-projecting monitors.

16. The modular shelter (1) according to claim 15, characterized in that a transparent or translucent material is glass, switchable glass and/or plastic.

17. The modular shelter (1) according to claim 16, characterized in that the glass is safety glass and consists of a single-layer safety glass pane or a multilayer safety glass laminated pane.

18. A hatch (11) for a shelter, comprising a partition wall (11.1) and two opening flaps (11.2) which are arranged on two different sides of the partition wall (11.1) and which allow access to a sliding tray (11.3) movable back and forth between the opening flaps (11.2), characterized in that in an interior space of the sliding tray (11.3) at least one surface irradiation device (11.4) and/or at least one tray irradiation device (11.5) for irradiating the interior space of the sliding tray (11.3) with UVC rays is arranged, and in that a bottom surface of the sliding tray (11.3) is formed from UV-permeable glass or UV-permeable plastic.

19. The hatch (11) according to claim 18, characterized in that the sliding tray (11.3) can be moved electromechanically and/or mechanically.

20. The hatch according to claim 18, characterized in that the at least one surface irradiation device (11.4) and/or the at least one tray irradiation device (11.5) irradiate the interior space of the sliding tray (11.3) with UVC radiation having a wavelength above 222 nm when the opening flaps (11.2) are closed.
